(19) United States
(12) Patent Application Publication
(10) Pub. No.: US 2018/0279661 A1
(43) Pub. Date: Oct. 4, 2018

(54) COMPOSITIONS COMPRISING MEDIUM CHAIN TRIGLYCERIDES

(71) Applicant: JUMP NATURAL INC., Ottawa (CA)

(72) Inventors: Audrey Sommerfeld, Hermosa Beach, CA (US); Steven Witherly, Valencia, CA (US); Caroline Witherly, Valencia, CA (US)

(21) Appl. No.: 15/940,590

(22) Filed: Mar. 29, 2018

Related U.S. Application Data
(60) Provisional application No. 62/478,705, filed on Mar. 30, 2017.

(51) Int. Cl. A23L 33/12; A23L 33/105; A23L 33/00; A61K 31/20

(52) U.S. Cl. CPC A23L 33/12; A23L 33/105; A23L 33/30; A61K 31/20; A23V 2250/1586; A23V 2200/3322; A23V 2250/1944; A23V 2250/62; A23V 2250/2108; A23V 2002/00

(57) ABSTRACT

Provided herein are compositions including medium chain triglycerides (MCT), methods for the preparation therefore, and methods for the use thereof. Compositions may include, for example, MCT, green coffee bean extract, and Irvingia extract. Compositions described herein may be for use in, for example, weight management in a subject in need thereof. Compositions may, in certain examples, be provided as a powder mix, which may be for use as a creamer or additive for mixing with variety of beverages and/or foods.

This application claims the benefit of U.S. provisional application No. 62/478,705 filed Mar. 30, 2017, the entire content of which is expressly incorporated herein by reference thereto.

### FIELD

The present application relates to compositions comprising medium chain triglyceride and methods of using the compositions.

### BACKGROUND

Obesity can be defined as an excessive accumulation of fat in the adipose tissue. It is well known that obesity increases the likelihood of various diseases such as type 2 diabetes mellitus, cardiovascular disease, stroke and some types of cancers. During the past decades there has been a dramatic increase in obesity worldwide. Obesity in humans is treated by a variety of means including surgical procedures, diet therapy, behavior modification, and medication.

Ketogenic diet, a high-fat, adequate-protein, low-carbohydrate diet that has been used primarily to treat refractory epilepsy in children, has gained popularity as a weight loss intervention. In essence, a ketogenic diet allows a human body to go into a metabolic state called ketosis or ketogenesis, which produces ketone bodies in large quantities by the liver in response to low blood glucose and insulin. Ketone bodies, acetone, acetoacetate (AcAc) and D-β-hydroxybutyrate (βHB), are respiratory fuels that are endogenously produced by the body from mobilised fat. Ketone bodies feed all the cells of a human body, but in particular are a preferred fuel for the brain. It is believed that the evolutionary effect of ketone bodies is therefore to spare carbohydrate reserves, and muscle protein, whilst themselves being an alternative energy source. However, it is difficult to maintain a ketogenic diet long term.

Various ketone supplements are available on the market, which may help control hunger, enhance energy, burn fat and support cognitive (brain) function. However, these available ketone supplements have been shown to either lack efficacy (for example, raspberry ketones) or contain high amounts of sodium and potassium which can spike blood pressure (for example, βHB salts). In addition, ketone supplements (both ketone salts and medium chain triglycerides oils) may also be harsh on stomach or require significant amount of sugar additives to mask the bitter taste of these supplements.

Therefore, there exists a need for improved dietary complements providing ketone bodies for both efficacy and ease of digestion.

### SUMMARY

In one aspect, there is provided a composition comprising medium chain triglycerides (MCT) and a natural sugar substitute.

In an embodiment of the composition as described herein, the MCT are triglycerides of C6, C8, C10 and/or C12 fatty acids in which the proportions of these fatty acids range in weight percent about from 0% to 2%, 50% to 65%, 30% to 45%, and 0%/o to 3%, respectively.

In an embodiment of the composition as described herein, the C6 fatty acid is caproic acid, C8 fatty acid is caprylic acid, C10 fatty acid is capric acid and C12 fatty acid is lauric acid and wherein the ratio of caprylic acid to capric acid is about 60:40. In certain embodiments, the ratio may be about 60:40 by weight ratio, or by mole ratio.

In an embodiment of the composition as described herein, the MCT comprises choline.

In an embodiment of the composition as described herein, the MCT are spray dried onto a fiber carrier.

In an embodiment of the composition as described herein, the fiber carrier is FIBERSOL®-2.

In an embodiment of the composition as described herein, the natural sugar substitute is isomaltulose. In yet another embodiment, the natural sugar substitute may be, for example, PALATINOSE®.

In an embodiment, the composition as described herein comprises about 30% to about 65%, for example about 55%, of the MCT. In certain embodiments, the composition as described herein may comprise about 30% to about 65% w/w, for example about 55% w/w, of the MCT. In certain embodiments, the composition as described herein may comprise about 30% to about 65% w/v, for example about 55% w/v, of the MCT.

In an embodiment, the composition as described herein comprises about 0.1% to about 10%, for example about 0.1% to about 1.0%, of the natural sugar substitute. In certain embodiments, the composition as described herein may comprise about 0.1% to about 10% w/w, for example about 0.1% to about 1.0% w/w, of the natural sugar substitute. In certain embodiments, the composition as described herein may comprise about 0.1% to about 10% w/v, for example about 0.1% to about 1.0% w/v, of the natural sugar substitute.

In an embodiment, the composition as described herein further comprises at least one of a green coffee bean extract, an Irvingia extract and chromium. In certain embodiment, the Irvingia extract may be Irvingia gabonensis extract. In certain embodiment, the chromium may be chromium picolinate. In certain embodiments, the green coffee bean extract may be, for example, SVETOL®.

In an embodiment, the composition as described herein further comprises a green coffee bean extract, an Irvingia extract and chromium.

In an embodiment, the composition as described herein is free of artificial colors, preservatives, flavors or sweeteners.

In an embodiment, the composition as described herein is formulated as a powder.

In another aspect, the present invention provides a dietary supplement comprising the composition as described herein.

In an embodiment, the dietary supplement as described herein is added to a beverage or food.

In an embodiment of the dietary supplement as described herein, the beverage is coffee or tea.

In another aspect, the present invention provides a method of weight management comprising administering the composition as described herein or the dietary supplement as described herein to a subject. In yet another aspect, there is provided herein a use of a composition or dietary supplement as described herein for weight management in a subject in need thereof. In still another aspect, there is provided herein a use of a composition as described herein in the manufacture of a dietary supplement for weight management in a subject in need thereof.

In an embodiment, the method or use or dietary supplement as described herein inhibits sugar and fat absorption in the subject.

In an embodiment, the method or use or dietary supplement as described herein enhances the energy level of the subject.

In an embodiment, the method or use or dietary supplement as described herein supports brain function.

In another embodiment, there is provided herein a composition comprising medium chain triglycerides (MCT), green coffee bean extract, and Irvingia extract.

In yet another embodiment, the MCT may be spray dried onto a fiber carrier.

In still another embodiment, the green coffee bean extract may comprise chlorogenic acid. In yet another embodiment, the green coffee bean extract may be, for example, SVETOL®.

In another embodiment, the green coffee bean extract may be decaffeinated.

In yet another embodiment, the Irvingia extract may comprise Irvingia gabonensis extract. In still another embodiment, the Irvingia extract may comprise Irvingia gabonensis seed extract.

In another embodiment, the composition may further comprise a natural sugar substitute.

In yet another embodiment, the natural sugar substitute may be or comprise isomaltulose.

In still another embodiment of the composition, the composition may comprise about 0.1% to about 10%, for example 0.1% to about 1.0%, of the natural sugar substitute. In certain embodiments, the composition may comprise about 0.1% to about 10% w/w, for example about 0.1% to about 1.0% w/w, of the natural sugar substitute. In certain embodiments, the composition may comprise about 0.1% to about 10% w/v, for example about 0.1% to about 1.0% w/v, of the natural sugar substitute.

In yet another embodiment, the MCT may comprise C₈ and C₁₀ fatty acids, and a ratio of the C₈ to C₁₀ fatty acids may be about 60:40. In certain embodiments, the ratio may be about 60:40 by weight ratio, or by mole ratio.

In still another embodiment, the MCT may comprise triglycerides of C6, C8, C10 and/or C12 fatty acids in which the proportions of these fatty acids range in weight percent about from 0% to 2%, 50% to 65%, 30% to 45%, and 0% to 3%, respectively.

In yet another embodiment, the C6 fatty acid may be caproic acid, C8 fatty acid may be caprylic acid, C10 fatty acid may be capric acid and C12 fatty acid may be lauric acid.

In still another embodiment, the ratio of caprylic acid to capric acid may be about 60:40.

In yet another embodiment, the MCT may comprise choline.

In another embodiment, the composition may comprise about 30% to about 65% of the MCT. In certain embodiments, the composition may comprise about 30% to about 65% w/w, for example about 55% w/w, of the MCT. In certain embodiments, the composition may comprise about 30% to about 65% w/v, for example about 55% w/v, of the MCT.

In still another embodiment, the composition may further comprise chromium picolinate.

In yet another embodiment, the composition may further comprise one or more of Monk fruit extract, Rebaaudioside, or natural flavors.

In still another embodiment, the composition may further comprise French vanilla cream flavoring.

In yet another embodiment, the composition may be free of artificial sweeteners, high fructose corn syrup, or both.

In still another embodiment, the composition may be in dried powder form. In yet another embodiment, the composition may be in dried powder form, which may form a substantially homogenous mixture or emulsion when mixed with water or aqueous beverage.

In another embodiment, there is provided herein a dietary supplement comprising a composition as described herein.

In yet another embodiment, the dietary supplement may be a dietary supplement which is added to a beverage or food. In certain embodiments, the beverage may be coffee or tea, for example.

In another embodiment, there is provided herein a use of the composition or dietary supplement as described herein for weight management in a subject in need thereof.

In certain embodiments, the composition or dietary supplement may inhibit sugar and fat absorption in the subject.

In yet another embodiment, the composition or dietary supplement may enhance an energy level of the subject.

In still another embodiment, the composition or dietary supplement may support brain function.

In yet another embodiment, there is provided herein a use of a composition as described herein in the manufacture of a dietary supplement for weight management in a subject in need thereof.

In another embodiment, the dietary supplement may inhibit sugar and fat absorption in the subject.

In yet another embodiment, the dietary supplement may enhance an energy level of the subject.

In still another embodiment, the dietary supplement may support brain function.

In another embodiment, the composition or dietary supplement may be for administration to the subject two or more times per day.

In still another embodiment, the composition or dietary supplement may be for administration orally.

In yet another embodiment, the composition or dietary supplement may be mixed with water, a hot or cold beverage, a shake, yogurt, cottage cheese, or another food.

In still another embodiment, the green coffee bean extract and Irvingia extract may function as ketogenesis facilitators by increasing fat release and keeping blood sugar levels low.

In another embodiment, the composition or dietary supplement may reduce hunger, support ketones, burn fat, or any combination thereof.

In another embodiment, there is provided herein a method for preparing a dietary supplement, said method comprising:
spray drying medium chain triglycerides (MCT) onto a fiber carrier; and
mixing the spray-dried MCT with green coffee bean extract and Irvingia extract, so as to provide the dietary supplement in a substantially homogenous powder form.

In another embodiment, there is provided herein a method of weight management, comprising:
administering a composition or dietary supplement as described herein to a subject in need thereof.

In another embodiment, the method inhibits sugar and fat absorption in the subject.

In still another embodiment, the method enhances an energy level of the subject.

In yet another embodiment, the method supports brain function.

In still another embodiment, the composition or dietary supplement is administered to the subject two or more times per day.

In yet another embodiment, the composition or dietary supplement is administered orally.

In yet another embodiment, the composition or dietary supplement is mixed with water, a hot or cold beverage, a shake, yogurt, cottage cheese, or another food.

In still another embodiment, the green coffee bean extract and Irvingia extract may function as ketogenesis facilitators by increasing fat release and keeping blood sugar levels low.

In yet another embodiment, the method may reduce hunger, support ketones, burn fat, or any combination thereof.

Other aspects and features of the present invention will become apparent to those of ordinary skill in the art upon review of the following description of specific embodiments of the invention in conjunction with the accompanying figures.

### DETAILED DESCRIPTION

Compositions and methods of the present invention relate to weight management. It has been found that a combination of medium chain triglycerides (MCT) and a natural sugar substitute may formulate a source of ketone bodies that is low in sugar, low in salt, gentle to the stomach and has a desirable taste.

Compositions provided herein may fuel circulating ketone bodies, support fat metabolism, enhance energy, and/or support brain function.

It is to be understood that extraction from a plant may be performed using conventional techniques such as phenolic extraction, from any part of the plant such as the flower, seed, fruit, root, tubercle, leaf, pericarp and rhizome. The extraction solvents may be chosen from, without limitation, water, propylene glycol, butylene glycol, glycerol, PEG-6 caprylic/capric glycerides, polyethylene glycol, methyl and/or ethyl esters, diglycols, cyclical polyols, ethoxylated or propoxylated diglycols, alcohols (such as methanol, ethanol, propanol, and butanol) and any mixture of these solvents. Plant extracts may also be obtained by other processes such as maceration, simple decoction, lixiviation, reflux extraction, super-critical extraction with CO₂, ultrasound or microwave extraction, or counter-current techniques. This list is not restrictive.

As used herein, a “subject” may include a mammal, such as farm animals, sport animals, pets, primates, horses, dogs, cats, mice, and rats, or humans.

As used herein, “administering/administration” means that a composition is delivered to a subject in such a way that it can achieve a desired purpose. The amount of compositions administered may vary depending upon factors such as the weight and the overall health of the subject.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

Medium-chain triglycerides (MCT) are esters derived from glycerol and 3 fatty acids with 5 to 12 carbon chain length, and may be represented by the formula of:

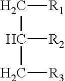

wherein the R1, R2, and R3 esterified to the glycerol backbone are each independently fatty acids having 5 to 12 carbon chains.

In the gastro-intestinal system, MCT are hydrolyzed rapidly to medium-chain fatty acids (MCFA) which are transported to the liver via the portal circulation where they are oxidized to ketones. In contrast, long-chain triglycerides (LCT), commonly found in Western diets, are absorbed in the intestine and incorporated to chylomicrons, where they undergo reesterification to reach the bloodstream via the lymphatic system. Within the bloodstream, lipoprotein lipases hydrolyze the LCT into smaller molecules such as monoglycerides and fatty acids, or they can be captured by diverse tissues such as muscle tissues and undergo oxidation, or stored in adipose tissue.

It is believed that, in addition to faster metabolism and less storage in adipocytes, MCT promote increased total energy expenditure of an individual. Thus, MCT may aid in control of body weight and reduce the harmful effects of obesity.

In some embodiments, the MCT are triglycerides of C6, C8, C10 and/or C12 fatty acids in which the proportions of these fatty acids range in weight percent about from 0% to 2%, 50% to 65%, 30% to 45%, and 0% to 3%, respectively. The C6 fatty acid may be caproic acid, the C8 fatty acid may be caprylic acid, the C10 fatty acid may be capric acid and the C12 fatty acid may be lauric acid.

It is believed that hydrolysis of MCT in stomach may cause upset stomach. It has been found that the ratio of C8 fatty acid to C10 fatty acid in MCT should be in the range of about 75:25 to about 45:55 in order to reduce the likelihood of MCT causing upset stomach. In some embodiments, the ratio of C8 fatty acid to C10 fatty acid in MCT is about 60:40.

In some embodiments, the MCT are spray dried onto a fiber carrier. Without being limited by theory, it is believed that the fiber carrier may prevent hydrolysis of MCT in stomach, thus reducing the likelihood of upset stomach caused by MCT. One embodiment of the fiber carrier is FIBERSOL®-2, a clinically tested fiber that blocks fat absorption and stimulates probiotics such as bifidobacterium.

In some embodiments, the MCT comprise choline. Choline is a vitamin-like essential nutrient involved in many physiological processes, including normal metabolism, transport of lipids, and brain function. Choline may also support heart health and other processes in the body.

A natural sugar substitute is a natural sugar or sugar alcohol that does not raise the level of blood glucose in a human body appreciably. Suitable natural sugar substitutes may include, but are not limited to, isomaltulose, erythritol, trehalose, allulose, tagatose, isomalt, lactitol, mannitol, sorbitol, xylitol, and fructose.

In some embodiments, the natural sugar substitute is isomaltulose. Isomaltulose is a natural sweetener derived from sugar beets. It has been clinically studied and tested, and shown to enter the blood stream in a sustained manner so that it does not spike insulin like common sugars. Studies also show that isomaltulose can support the body's fat burning process.

It is believed that the combination of MCT and a natural sugar substitute provides a source of ketone bodies that is quickly metabolized, gentle on stomach and low in sugar and salt, without artificial sweeteners or junk fillers.

In some embodiments, compositions provided herein comprise about 30% to about 65%, for example about 55%, of the MCT. In some embodiments, compositions provided herein comprise about 0.1% to about 10%, for example about 0.1% to about 1.0% of the natural sugar substitute.

In some embodiments, compositions provided herein further comprise an Irvingia extract. Irvingia gabonensis is a tree native to West Africa. Its fruit is also known as African Mango. The seed has been studied, and may help support healthy cholesterol levels as well as help promote weight loss. There also have been several clinical trial studies showing that Irvingia extracts can delay gastric emptying, decrease sugar absorption, decrease fat absorption, decrease the size of fat cells, activate PPAR gamma to reduce body fat, and decrease leptin and increase adiponectin both of which are believed to reverse metabolic syndrome.

In some embodiments, compositions provided herein further comprise a green coffee bean extract. Green coffee bean extracts are derived from green, unprocessed coffee beans (beans that have not been roasted). Green coffee bean extracts naturally contain chlorogenic acid, a powerful antioxidant. Studies show that an adequate intake of a green coffee bean extract may help promote weight loss. In some embodiments, green coffee bean extracts are decaffeinated. There are several commercially available green coffee bean extracts, such as GCBFIT™ sold by HerbaKraft, Inc. In certain embodiments, the green coffee bean extract may comprise SVETOL®, for example.

In some embodiments, compositions provided herein do not comprise harsh stimulants, such as Ephedra or bitter orange.

In some embodiments, compositions provided herein further comprise chromium, an essential nutrient (trace element) required for the normal metabolism of carbohydrate, protein and fats.

In some embodiments, compositions provided herein further comprise at least one of, at least two of, or all of an Irvingia extract, a green coffee bean extract and chromium. In certain embodiments, the chromium may be provided as chromium picolinate, for example.

In some embodiments, compositions provided herein comprise MCT, isomaltulose, an Irvingia extract, a green coffee bean extract and chromium, wherein the MCT are triglycerides of C6, C8, C10 and/or C12 fatty acids in which the proportions of these fatty acids range in weight percent about from 0% to 2%, 50% to 65%, 30% to 45%, and 0% to 3%, respectively and the ratio of C8 fatty acid to C10 fatty acid is about 60:40. It is believed such compositions may simultaneously induce satiety, inhibit sugar and fat absorption, enhance fat burning and lipolysis, and activate hormonal pathways like PPAR gamma, leptin and adiponectin, without harsh stimulants, high levels of salts and sugars, junk fillers, or artificial colors, preservatives, flavors or sweeteners.

In some embodiments, compositions provided herein are used to facilitate weight loss, as a brain tonic, to enhance athletic performance, to help prevent diseases related to obesity, as an adjunct to a ketogenic diet, and other uses associated with improved metabolic health. Compositions provided herein may elevate blood ketones to a level that would be considered a state of nutritional ketosis.

Administration of compositions provided herein may be performed with or without dietary restriction. In some embodiments, the subject follows a ketogenic diet that restricts intake of carbohydrates and protein during the period of administration of compositions provided herein. A ketogenic diet may restrict the dietary intake to a ratio of about 65% fat, 25% protein, and 10% carbohydrates. In some embodiments, the subject is not on a diet.

Compositions provided herein may be administered once per day, twice per day, or three times per day to a subject desiring to promote and/or sustain a state of ketosis and/or to have an elevated level of ketone bodies.

Compositions provided herein may be administered to a subject by a number of suitable methods known in the art. Examples of suitable methods include: (1) intramuscular, intradermal, intraepidermal, or subcutaneous administration, (2) oral administration, and (3) topical application (such as ocular, intranasal, and intravaginal application). In some embodiments, the preferred route of administration of compositions provided herein is oral.

Compositions provided herein may be delivered as a powdered mixture, as a ready-to-drink liquid, in hard or soft gelatin caps, as hard-pressed tablets, concentrated gels, or any other dosage form known to those trained in the art.

In some embodiments, compositions provided herein are formulated together with a flavoring agent as dietary supplements useful for weight loss and improving a subject's general health, without harsh stimulants, high levels of salts and sugars, junk fillers, or artificial colors, preservatives, flavors or sweeteners. In some embodiments, compositions provided herein are formulated as powders, such as coffee creamers, to be added to beverages such as coffee, tea, protein shake and yogurt, or food such as cookies, protein bars and cottage cheese.

### Example 1—Powder Mix MCT Composition

MCT purchased from SensoryEffects®, a division of BALCHEM, isomaltulose, natural flavors, green coffee bean extract, Irvingia extract and chromium picolinate were blended into a powder mix having a vanilla cream flavor. Each serving size of this powder mix in the amount of 9 g contains 60 calories, 5 g of the MCT, 3 g of total carbohydrate, 2 g of fiber and 5 mcg of chromium.

### Example 2—Dietary Supplement Powder, and Preparation Thereof

In this example, a dietary supplement powder composition comprising medium chain triglycerides (MCT), green coffee bean extract, and Irvingia extract was prepared. The dietary supplement powder was formulated for use as a creamer to be added to water or other beverages, and was formulated for administration to a subject two or more times daily. The dietary supplement did not contain high-fructose corn syrup or other corn syrups, to avoid raising blood sugar levels.

In this dietary supplement example, without wishing to be bound by theory, the green coffee bean extract and Irvingia extract may function as ketogenesis facilitators by increasing fat release and keeping blood sugar levels low.

The MCT of the composition may, without wishing to be bound by theory, support circulating ketones in the body. The MCT of the composition was spray dried onto a fiber carrier, which may reduce the likelihood of upset stomach. The MCT comprised C8 and C10 fatty acids, and a ratio of the C8 to C10 fatty acids was about 60:40 by weight. The MCT comprised triglycerides of C6, C8, C10 and/or C12 fatty acids in which the proportions of these fatty acids range in weight percent about from 0% to 2%, 50% to 65%, 30% to 45%, and 0% to 3%, respectively. The C6 fatty acid was caproic acid, C8 fatty acid was caprylic acid, C10 fatty acid was capric acid and C12 fatty acid was lauric acid, and the ratio of caprylic acid to capric acid was about 60:40 by weight, which may also reduce the likelihood of upset stomach in certain examples.

The green coffee bean extract of the composition comprised chlorogenic acid (which may, without wishing to be bound by theory, promote weight loss), and the green coffee bean extract was decaffeinated. The Irvingia extract comprised Irvingia gabonensis seed extract, which may, without wishing to be bound by theory, support healthy cholesterol and/or weight. The composition further comprised the natural sugar substitute isomaltulose, a natural sweetener derived from sugar beets which is believed to enter the blood in a relatively sustained manner to avoid spiking insulin, and chromium picolinate (which may, without wishing to be bound by theory, support healthy blood sugar levels and/or fat utilization).

As well, the composition additionally comprised Monk fruit extract, Rebaaudioside, and natural flavors. The composition comprised French vanilla cream flavoring, and was free of artificial sweeteners and high fructose corn syrup.

The composition was provided in dried powder form. Upon mixing with water and other aqueous fluids, the dried powder formed a substantially homogenous mixture.

The dietary supplement of this example was prepared by:
spray drying the medium chain triglycerides (MCT) onto a fiber carrier; and
mixing the spray-dried MCT with green coffee bean extract and Irvingia extract, so as to provide the dietary supplement in a substantially homogenous powder form.

As will be understood, the spray drying of the MCT onto a fiber carrier may be performed using any suitable spray-drying technique (or functional equivalent) known to the person of skill in the art having regard to the teachings herein. Without wishing to be bound by theory, providing the MCT in such form may prevent or reduce hydrolysis of MCT in the stomach following ingestion, which may reduce the likelihood of upset stomach caused by MCT. The fiber carrier may be any suitable fiber-based carrier known to the person of skill in the art having regard to the teachings herein. By way of illustrative example, in one embodiment the fiber carrier may be Fibersol®-2, a clinically tested fiber that blocks fat absorption and stimulates probiotics such as bifidobacterium.

The mixing may be achieved using any suitable mixing technique and/or apparatus known to the person of skill in the art having regard to the teachings herein, and may be performed so as to provide the dietary supplement in the form of a substantially homogenous mixture, which may be provided in, for example, a powder form amendable to mixing with water or other beverages.

### Example 3: Treatment of Subjects with the Dietary Supplement

Subjects desirous of losing weight were treated with a dietary supplement as described herein twice daily as part of a healthy diet and lifestyle.

At least some of the subjects taking the dietary supplement reported one or more of weight loss, reduced hunger, improved mood, improved mental clarity and focus, an easier time reaching/maintaining ketosis levels, and/or enhanced energy during treatment.

What is claimed is:

1. A composition comprising medium chain triglycerides (MCT) and a natural sugar substitute.

2. The composition according to claim 1, wherein the MCT are triglycerides of C6, C8, C10 and/or C12 fatty acids in which the proportions of these fatty acids range in weight percent about from 0% to 2%, 50% to 65%, 30% to 45%, and 0% to 3%, respectively.

3. The composition according to claim 2, wherein the C6 fatty acid is caproic acid, C8 fatty acid is caprylic acid, C10 fatty acid is capric acid and C12 fatty acid is lauric acid and wherein the ratio of caprylic acid to capric acid is about 60:40.

4. The composition according to claim 1, wherein the MCT are spray dried onto a fiber carrier.

5. The composition according to claim 1, wherein the natural sugar substitute is isomaltulose.

6. The composition according to claim 1, comprising about 30% to about 65% of the MCT, and about 0.1% to about 1.0% of the natural sugar substitute.

7. The composition according to claim 1 further comprising at least one of a green coffee bean extract, an Irvingia extract and chromium.

8. The composition according to claim 1 which is formulated as a powder.

9. A method of weight management comprising administering the composition of claim 1 to a subject in need thereof.

10. The method according to claim 9, which inhibits sugar and fat absorption in the subject; enhances an energy level of the subject; and/or supports brain function in the subject.

11. A composition comprising medium chain triglycerides (MCT), green coffee bean extract, and Irvingia extract.

12. The composition of claim 11, wherein the MCT are spray dried onto a fiber carrier.

13. The composition of claim 11, wherein the composition further comprises chromium, a natural sugar substitute such as isomaltose, or both.

14. The composition claim 11, wherein the MCT comprises C8 and C10 fatty acids, and a ratio of the C8 to C10 fatty acids is about 60:40.

15. The composition of claim 14, wherein the MCT are triglycerides of C6, C8, C10 and/or C12 fatty acids in which the proportions of these fatty acids range in weight percent about from 0% to 2%, 50% to 65%, 30% to 45%, and 0% to 3%, respectively, and the C6 fatty acid is caproic acid, C8 fatty acid is caprylic acid, C10 fatty acid is capric acid and C12 fatty acid is lauric acid.

16. The composition according to claim 11, comprising about 30% to about 65% of the MCT.

17. The composition of claim 11, in dried powder form, which forms a substantially homogenous mixture when mixed with water.

18. A method of weight management, comprising:
administering the composition according to claim 11 to a subject in need thereof.

19. The method according to claim 18, wherein the method inhibits sugar and fat absorption in the subject; enhances an energy level of the subject; supports brain function in the subject; reduces hunger, supports ketones; burns fat; and/or wherein the green coffee bean extract and Irvingia extract function as ketogenesis facilitators by increasing fat release and keeping blood sugar levels low.

20. A method for preparing a dietary supplement, said method comprising:
spray drying medium chain triglycerides (MCT) onto a fiber carrier, and
mixing the spray-dried MCT with green coffee bean extract and Irvingia extract, so as to provide the dietary supplement in a substantially homogenous powder form.
